(19) United States
(12) Patent Application Publication
(10) Pub. No.: US 2006/0009790 A1
(43) Pub. Date: Jan. 12, 2006

(54) Anti-backup mechanism for repeating multi-clip applier

(76) Inventors: Joseph W. Blake III, New Canaan, CT (US); Kenneth J. Lisk, Fairfield, CT (US)

Correspondence Address: Patrick J. Walsh Esq., 400 Main Street, Stamford, CT, 06901, US

(21) Appl. No.: 11/224,487

(22) Filed: Sep. 12, 2005

Related U.S. Application Data
(62) Division of application No. 10/315,969, filed on Dec. 10, 2002, now abandoned.

(51) Int. Cl. A61B 17/10

(52) U.S. Cl. 606/142

(57) ABSTRACT

An instrument for applying clips in surgery having an operating handle with operating components and a clip cartridge with clip applying mechanism. The handle operating components generate reciprocal linear motion imparted to the clip applying mechanism and accommodate rotation of the cartridge about a cartridge axis. An anti-backup mechanism constrains operating components to complete first and second strokes of reciprocal linear motion.

### PRIOR APPLICATION

This application is a division of U.S. patent application Ser. No. 10/315,969 filed Dec. 10, 2002, now abandoned.

### BACKGROUND OF THE INVENTION

The present invention relates to surgical clip appliers embodied as an instrument having a supply of clips for rapidly deploying several clips in closing severed blood vessels and other small fluid carrying ducts in surgical procedures. There are many different designs for surgical clip applicators for a variety of surgical procedures including both open surgery and laparoscopy in which a clipping appliance fits through a trocar tube into a body cavity where the clips are applied.

A surgical clip applicator comprises an operating handle and clip applying mechanism having an operating cycle in which operating levers are squeezed together and released. In this operating cycle, a clip is applied in surgery and the clip applicator jaws are reloaded with a single clip from a clip supply channel for clip application in the next cycle. The applicator provides a moveable clip supply channel containing a line of clips that are released seriatim. The supply channel integrates a clip pusher and an escapement or clip stop spring in a single unit.

A well-known hazard with clip appliers is a condition of releasing a partially closed clip in a surgical site. This condition results when operating handles are given a partial pull or closing and then released. The partial pull crimps but does not close a clip located in the instrument jaws. When partially pulled handles of some older instruments are released, the instrument jaws re-open and the partially closed clip falls from the jaws into the surgical site.

This invention provides an anti-backup mechanism for a repeating multi-clip applier to prevent this operating hazard.

### SUMMARY OF THE INVENTION

A preferred embodiment of repeating multi-clip applier according to the present invention comprises an instrument having an operating handle housing and a removable, fully rotatable and disposable clip applying cartridge. A full squeeze and release of operating handles applies a clip to a surgical site and reloads another clip into clip applying jaws of the instrument.

The operating handle housing accommodates an anti-backup mechanism to prevent a partial pull and release of the operating handles to prevent the hazard of a partially closed clip falling into the surgical site. The present invention prevents occurrence of this condition by means of an anti-backup mechanism to ensure that when the appliance handles are pulled, the handles must be given a full pull to execute a complete cycle of the clip applier mechanism. If a partial pull of the appliance handles occurs, the anti-backup mechanism retains or holds the clip applier mechanism in fixed position without possibility of backup or reverse. The anti-backup “hold” is released simply by giving the handles a complete pull. When a partial pull occurs and the anti-backup mechanism holds the clip applying mechanism in place, the instrument jaws remain partially closed holding a partially closed clip thereby preventing the clip from falling into a surgical site. The anti-backup mechanism also functions in the opposite, or release, motion of operating handles. That is, the operating handles when being released are constrained by the anti-backup mechanism to undergo a full release motion. The anti-backup mechanism prevents partial release and re-pull of the trigger thereby to prevent double loading of a clip into the crimping jaws, a condition that would jam the instrument.

Specific examples are included in the following description for purposes of clarity, but various details can be changed within the scope of the present invention.

### OBJECTS OF THE INVENTION

An object of the invention is to provide a clip applicator having an operating handle that provides anti-backup linear reciprocating motion of a clip applying cartridge.

Another object of the invention is to provide a clip applicator having an operating handle with an anti-backup mechanism that permits full forward and reverse strokes of a clip applying cartridge to avoid the hazard of releasing a partially crimped clip at a surgical site.

Another object of the invention is to provide a clip applicator having an operating handle with an anti-backup mechanism that permits full forward and reverse strokes of a clip applying cartridge to prevent double loading of a clip into the crimping jaws, a condition that would jam the instrument.

Other and further objects of the invention will become apparent with an understanding of the following detailed description of the invention or upon employment of the invention in practice.

### BRIEF DESCRIPTION OF THE DRAWING

A preferred embodiment of the invention has been chosen for detailed description to enable those having ordinary skill in the art to which the invention appertains to readily understand how to construct and use the invention and is shown in the accompanying drawing in which:

FIG. 1 is a side elevation view of the applicator of FIG. 1 with the operating handle housing partially in section and with handles in release position.

FIG. 2 is a side elevation view of the applicator of FIG. 1 with the operating handle housing partially in section and with handles in pull position.

FIG. 3 is a side elevation of a rotary translator.

FIG. 4a is a side elevation view of the rotary drum.

FIG. 4b is a longitudinal section view of the rotary drum of FIG. 4a.

FIG. 5 is a front elevation of anti-backup disc.

FIGS. 6 a-g are sequential views of anti-backup mechanism with disc in (a) rear groove, (b)&(c) between grooves, (d) in front groove, (e)&(f) between grooves, and (g) again in rear groove, and with arrows indicating directions of permitted and prevented movement of operating handle and cartridge mechanism.

### DETAILED DESCRIPTION OF THE PREFERRED EMBODIMENT

Referring to the drawing, a preferred embodiment of the repeating multi-clip applier 10 comprises operating handle housing 12 and clip applicator cartridge 14.

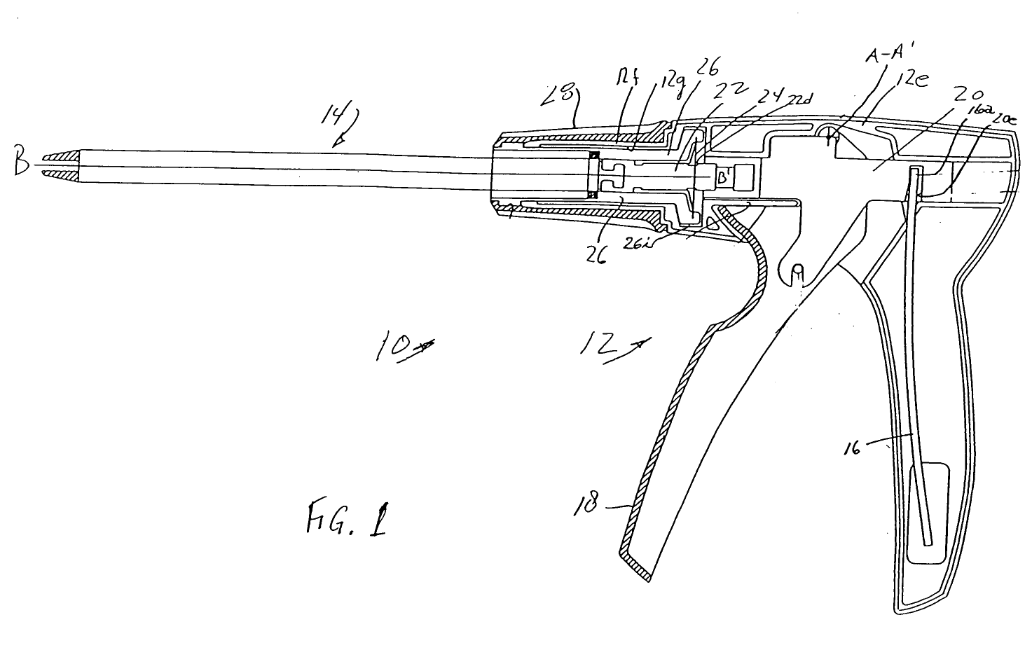

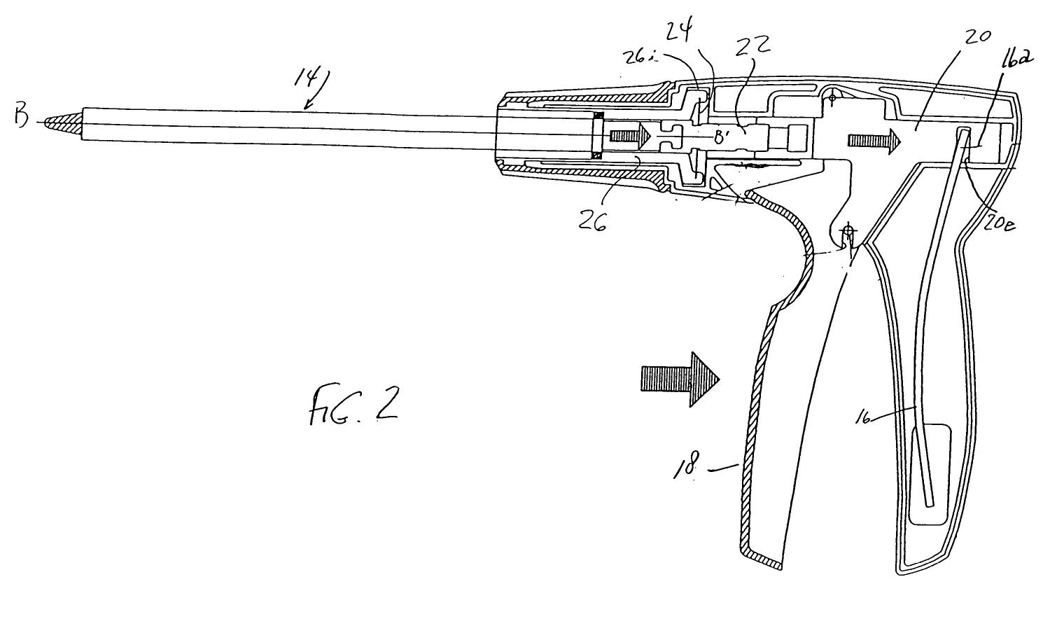

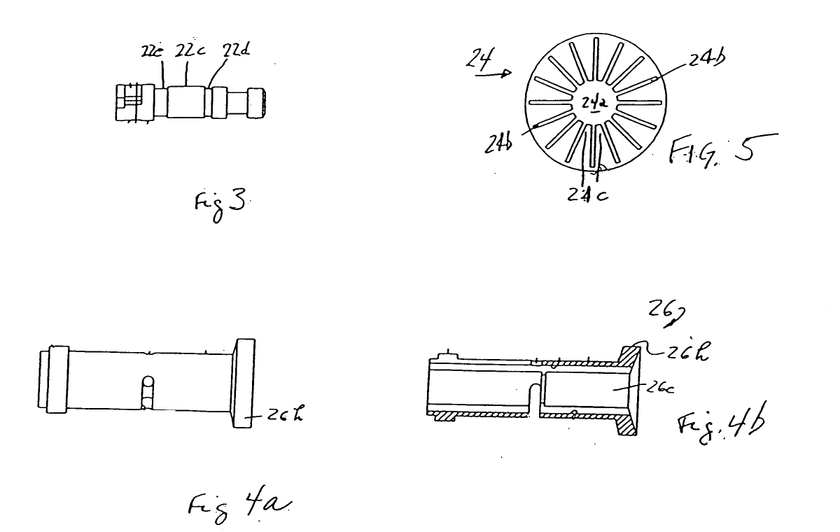

The operating handle housing 12 shown in FIGS. 1-2 comprises handle members including a depending grip 12c, a center section defining a central chamber 12e, and a forward cylindrical portion 12f defining a forward chamber 12g.

A trigger 18 for actuating applier mechanisms is mounted on the housing for pivotal movement about axis A-A′, normal to FIG. 1. The trigger includes a depending grip portion 18.

The trigger when pulled transmits motion to the clip cartridge mechanism (not shown) through the intermediation of fixed translator slide 20 and a rotary translator 22. The trigger acts against the forward bias of bar spring 16 with its end 16a held by translator slide recess 20a.

The rotary translator 22 (FIGS. 1, 2, and 3) forms a subassembly with an anti-backup mechanism 24, a rotatable drum 26, and a thumb wheel hub 28 which subassembly interconnects the fixed translator 20 and the clip cartridge 14 for performing the functions of transmitting reciprocating rectilinear motion with a fixed excursion, accommodating rotary motion of the clip cartridge, enabling mounting and disconnecting of the clip cartridge from the operating handle, and providing an anti-backup capability for the operating handle and cartridge mechanism.

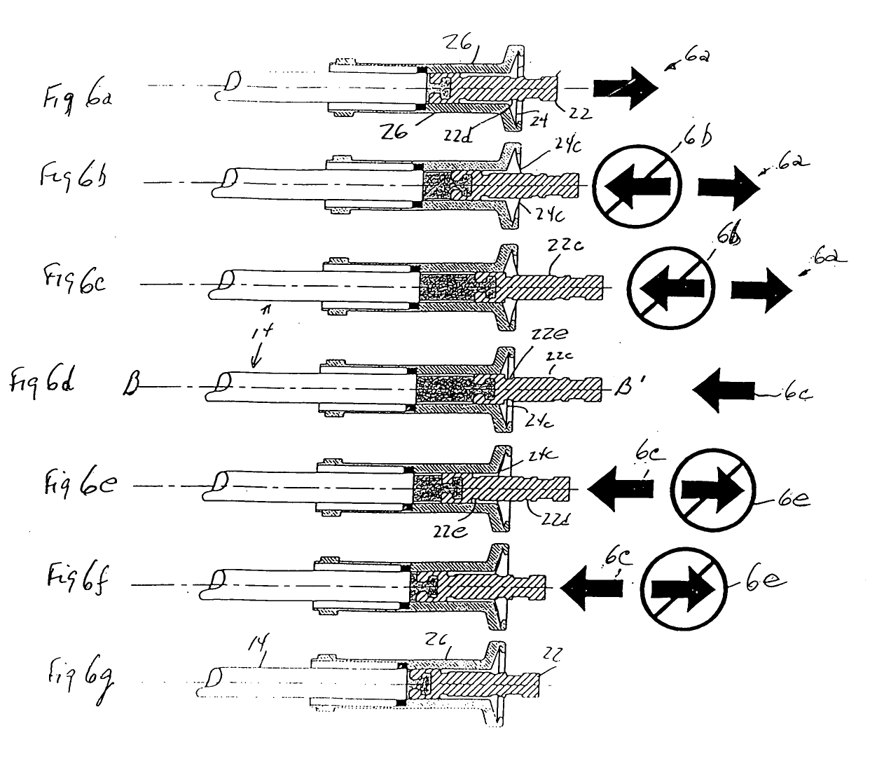

The rotary drum subassembly 22, 24, 26 comprises the rotary translator 22 positioned axially within the drum 26. An anti-backup disc 24 (FIGS. 2, 3 and 5), defined by an open center 24a and extending radially from the center to define a plurality of inwardly directed spring fingers 24c, fits onto the rotary translator 22 and is assembled to the enlarged rear end flange 26h of the rotary drum by means of a drum cap 26i (FIGS. 1 and 2). In normal position of the clip applicator with the trigger released, the anti-backup spring fingers 24c are located in the rear anti-backup groove as shown in FIG. 1.

The rotary drum subassembly is assembled into the forward chamber 12g of the operating handle housing. The center section 22c (FIG. 3) of the rotary translator shaft has spaced anti-backup grooves 22d, 22e with the distance between the grooves being approximately equal to the distance of reciprocating rectilinear motion of the fixed translator and equal to the rectilinear excursion of the clip applicator mechanism.

The operation of the anti-backup mechanism is illustrated in FIGS. 6a-g.

In the mechanism position of FIGS. 1 and 6a, the handle trigger 18 is in released position with the anti-backup disc 24 in registry with the rear anti-backup groove 22d of the rotary translator 22. When the trigger is pulled (FIG. 2) (for crimping and applying a clip at a surgical site), the rotary translator moves in the direction of arrow 6a. As the rotary translator continues movement, the spring fingers 24c of the disc engage the outer surface 22c of the rotary translator in the manner shown in FIGS. 6b-c. The anti-backup mechanism applied by the canted spring fingers 24c (FIGS. 6b-c) to the outer surface 22c of the rotary translator permits continued movement in the direction of arrow 6a and prevents movement in the opposite direction of arrow 6b. If a surgeon releases the trigger with less than a full pull stroke leaving anti-backup components in the position of FIG. 6c, for example, the anti-backup mechanism holds the rotary translator in position against the bias of bar spring 16 which tends to return the trigger to release position. In this FIG. 6c hold position, the applicator jaws retain the partially crimped clip preventing it from falling into a surgical site. A continuing pull of the trigger (in direction of arrow 6a) moves the rotary translator through the position of FIG. 6c to the position of FIG. 6d in which the spring fingers 24c enter the forward groove 22e.

In this position (FIG. 6d), the rotary translator may now be moved forward (by releasing the trigger and by force of return spring 16) in the direction of arrow 6c. In this forward movement, the spring fingers 24c are effective to allow continued forward movement while preventing movement in the direction of arrow 6e. If the handle trigger is held by a surgeon with components as in FIG. 6f, the anti-backup mechanism will prevent the surgeon from pulling the trigger in the direction of arrow 6e. The surgeon must allow full release of the trigger to component position of FIG. 6g. Direction of movement can be changed again when the spring fingers 24c enter the rear groove 22e as in FIG. 6g.

A pull on the trigger against the force of bar spring produces unitary rearward rectilinear movement of the fixed translator, the rotary translator passing through the stationary spring fingers of the anti-backup disc, and the puller bar emerging from within the cartridge casing until the trigger and fixed translator reach the end of travel and with the anti-backup disc spring fingers positioned at the front groove. The rearward excursion is now complete, and when the trigger is released, the bar spring urges the fixed translator forward until all components reach normal position.

In the event a pull on the trigger is released without reaching the full extent of rectilinear motion, the anti-back up spring fingers will not have reached their front groove remaining instead in contact with the outer surface of the rotary translator. The spring fingers in contact with outer surface function as a brake against the action of the bar spring tending to force the released components to return to normal position. In this partial pull condition of the trigger a clip has been crimped in the instrument jaws which clip will fall out of the jaws into a surgical site if the jaws reopen by return of the mechanism to normal position. So the anti-backup mechanism retains the instrument in “partial pull position” against the normalizing force of the bar spring and most importantly prevents fallout from the jaws of a partially crimped clip. The anti-backup device retaining action is removed simply by means of a full pull on the trigger causing the spring fingers to enter the forward groove where they can go “over center” thereafter permitting the rotary translator to pass through the spring fingers. It is to be noted that the anti-backup mechanism is effective in both directions. The anti-backup mechanism has effect when the trigger is released after a full pull so that if there is a “partial release” of the trigger, the trigger must nonetheless return to normal position with full release of the trigger before allowing the trigger to be pulled. The design requirement for full release achieved by the anti-backup mechanism prevents double loading of clips into cartridge jaws.

It is a further aspect of the anti-backup mechanism that the cartridge may be rotated on the B-B′ axis as the anti-backup mechanism holds the instrument in partial pull position enabling a surgeon to adjust cartridge or jaw position even after a partial pull has occurred.

Various changes may be made to the structure embodying the principles of the invention. The foregoing embodiments are set forth in an illustrative and not in a limiting sense. The scope of the invention is defined by the claims appended hereto.

We claim:

1. An anti-backup mechanism for a device having liner reciprocating motion through forward and reverse strokes, the anti-backup mechanism comprising a drum, a translator assembled with the drum for linear reciprocating movement through said strokes, the translator having first and second means defining the limits of reciprocating motion, and an anti-backup disc mounted on the drum and cooperating with the translator first and second means to ensure the forward and reverse strokes of linear excursion of the translator are completed without backup movement.
